(19) United States
(12) Patent Application Publication
(10) Pub. No.: US 2026/0345779 A1
(43) Pub. Date: Nov. 5, 2026

(54) KRAS/TP53 NEOANTIGEN SPECIFIC T CELL RECEPTORS

(71) Applicant: H. LEE MOFFITT CANCER CENTER AND RESEARCH INSTITUTE, INC., Tampa, FL (US)

(72) Inventors: Eric HAURA, St. Petersburg, FL (US); Ben CREELAN, Tampa, FL (US); Chao WANG, Tampa, FL (US)

(21) Appl. No.: 19/120,526

(22) PCT Filed: Oct. 13, 2023
(86) PCT No.: PCT/US2023/076804

§ 371 Date: Apr. 11, 2025

Related U.S. Application Data
(60) Provisional application No. 63/415,674, filed on Oct. 13, 2022.

(51) Int. Cl. A61K 40/42; A61K 39/395; A61K 40/11; A61K 40/15; A61K 40/17; A61K 40/31; A61K 40/32; A61P 35/00; C07K 14/705; C12N 5/0783; G01N 33/569

(52) U.S. Cl. CPC A61K 40/4201; A61K 39/3955; A61K 40/11; A61K 40/15; A61K 40/17; A61K 40/31; A61K 40/32; A61P 35/00; C07K 14/7051; C12N 5/0636; G01N 33/56972; C12N 2510/00

(57) ABSTRACT

Disclosed are T cell receptors (TCRs) specific for one or more neoantigens and T cells engineered to express said TCRs as well as methods of their use for treating cancer.

### II. CROSS REFERENCE TO RELATED APPLICATIONS

This application claims the benefit of U.S. Provisional Application No. 63/415,674, filed on Oct. 13, 2022, which is incorporated herein by reference in its entirety.

### I. STATEMENT OF GOVERNMENT SUPPORT

This invention was made with government support under Grant No. CA076292 awarded by National Institutes of Health. The government has certain rights in the invention.

### III. REFERENCE TO SEQUENCE LISTING

The sequence listing submitted on Oct. 13, 2023, as an .XML file entitled “10110-416WO1_ST26.xml” created on Oct. 12, 2023, and having a file size of 29,866 bytes is hereby incorporated by reference pursuant to 37 C.F.R. § 1.52 (e) (5).

### IV. BACKGROUND

Neoantigens are antigens created by non-synonymous somatic mutations and recognized by unique T cell receptor (TCR) clonotypes of CD8 or CD4. Studies into neoantigens have shown that some neoantigens that have been able to be identified may induce the durable remissions >1 decade via adoptive cell transfer. Once identified as a target, a neoantigen alone is not necessarily the end of the therapeutic discovery as TCRs can be designed that recognize neoantigens. Thus, what are needed are convenient and economic methods to identify relevant neoantigens for clinical applications is urgently needed.

### V. SUMMARY

Disclosed are TCRs that recognize one or more neoantigens and methods of their use.

In one aspect, disclosed herein are T cell receptors (TCRs) that recognize one or more neoantigens (such as, for example, the TP 53^{G244C} HLA A*11:01 neoantigen SSCMCGMNRR (SEQ ID NO: 32) or the KRAS^{G12V} HLA A*11:01 neoantigen VVGAVGVGK (SEQ ID NO: 33)), wherein the TCR comprises a TCRβ comprising the sequence CASEGDGRASNEQFF (SEQ ID NO: 1), CASIRLRGNTDTQYF (SEQ ID NO: 2), CASSRPWELQETQYF (SEQ ID NO: 4), CASRYHSGQGLQETQYF (SEQ ID NO: 3), CASSLVGSGRGGTDTQYF (SEQ ID NO: 5), CASAPGGNSPLHF (SEQ ID NO:6), CASSPGPNSPLHF (SEQ ID NO:7), CAREGDGGASNEQFF (SEQ ID NO:8), CARGGDGRASNEQFF (SEQ ID NO: 9), CASSVSGVTDTQYF (SEQ ID NO: 13), CASSLVGGYQETQYF (SEQ ID NO: 14), CASSFRTGDFSGANVLTF (SEQ ID NO: 15), CASSSPGISYEQYF (SEQ ID NO: 16), CASSFGMNTEAFF (SEQ ID NO: 17), CAISPGTYYGYTF (SEQ ID NO: 18), CASSPRDSNYGYTF (SEQ ID NO: 19), CASSVSVGVTDTQYF (SEQ ID NO: 20), CASSLTAGRALHF (SEQ ID NO: 21), or CASSTGGNSPLHF (SEQ ID NO: 22).

Also disclosed herein are T cell receptors of any preceding aspect, wherein the TCR further comprises a TCRα comprising the sequence CAGSGAGSYQLTF (SEQ ID NO: 10), CAEGSSASKIIF (SEQ ID NO: 11), CAMREGKGGGYNKLIF (SEQ ID NO: 12), CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), CAAKMNRDDKIIF (SEQ ID NO: 27), CALVGFNKFYF (SEQ ID NO: 28), CAMIPPPSVGFGNVLHC (SEQ ID NO: 29), CAVEGGGTSYGKLTF (SEQ ID NO: 30), or CAASDDSGGYQKVTF (SEQ ID NO: 31).

In one aspect, disclosed herein are T cells comprising the TCR of any preceding aspect.

In one aspect, disclosed herein are T cells (such as, for example, a tumor infiltrating lymphocyte (TIL), chimeric antigen receptor (CAR) T cell, or marrow infiltrating lymphocyte (MIL)) comprising the TCR of any preceding aspect.

Also disclosed herein are methods of treating, inhibiting, reducing, decreasing, ameliorating, and/or preventing a cancer and/or metastasis in a subject comprising administering to the subject a TCR, T cell, CAR T cell, TIL, and/or MIL of any preceding aspect. For example disclosed herein are methods of treating a cancer in a subject comprising administering to the subject a T cell, CAR T cell, TIL, and/or MIL comprising a TCR that recognizes a neoantigen including, but not limited to, neoantigens comprising the TP53^{G244C} sequence SSCMCGMNRR (SEQ ID NO: 32) or the KRAS^{G12V} HLA A*11:01 neoantigen VVGAVGVGK (SEQ ID NO: 33). For example, disclosed herein are methods of treating, inhibiting, reducing, decreasing, ameliorating, and/or preventing a cancer and/or metastasis in a subject comprising administering to the subject a TCR, T cell, CAR T cell, TIL, and/or MIL of any preceding aspect. For example disclosed herein are methods of treating a cancer in a subject comprising administering to the subject a T cell, CAR T cell, TIL, and/or MIL comprising a TCR a TCRβ comprising the sequence CASEGDGRASNEQFF (SEQ ID NO: 1), CASIRLRGNTDTQYF (SEQ ID NO: 2), CASSRPWELQETQYF (SEQ ID NO: 4), CASRYHSGQGLQETQYF (SEQ ID NO: 3), CASSLVGSGRGGTDTQYF (SEQ ID NO: 5), CASAPGGNSPLHF (SEQ ID NO:6), CASSPGPNSPLHF (SEQ ID NO:7), CAREGDGGASNEQFF (SEQ ID NO:8), CARGGDGRASNEQFF (SEQ ID NO: 9), CASSVSGVTDTQYF (SEQ ID NO: 13), CASSLVGGYQETQYF (SEQ ID NO: 14), CASSFRTGDFSGANVLTF (SEQ ID NO: 15), CASSSPGISYEQYF (SEQ ID NO: 16), CASSFGMNTEAFF (SEQ ID NO: 17), CAISPGTYYGYTF (SEQ ID NO: 18), CASSPRDSNYGYTF (SEQ ID NO: 19), CASSVSVGVTDTQYF (SEQ ID NO: 20), CASSLTAGRALHF (SEQ ID NO: 21), or CASSTGGNSPLHF (SEQ ID NO: 22) and/or a TCRα comprising the sequence CAGSGAGSYQLTF (SEQ ID NO: 10), CAEGSSASKIIF (SEQ ID NO: 11), CAMREGKGGGYNKLIF (SEQ ID NO: 12), CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), CAAKMNRDDKIIF (SEQ ID NO: 27), CALVGFNKFYF (SEQ ID NO: 28), CAMIPPPSVGFGNVLHC (SEQ ID NO: 29), CAVEGGGTSYGKLTF (SEQ ID NO: 30), or CAASDDSGGYQKVTF (SEQ ID NO: 31).

In one aspect, disclosed herein are methods of treating, inhibiting, reducing, decreasing, ameliorating, and/or preventing a cancer and/or metastasis of any preceding aspect, wherein the neoantigen recognized by the TCR is from a KRAS^{G12V} mutant cancer (such as, for example, VVGAVGVGK (SEQ ID NO: 33)). For example, the TCR can comprise a TCRβ comprising the sequence CASEGDGRASNEQFF (SEQ ID NO: 1), CASIRLRGNTDTQYF (SEQ ID NO: 2), CASSRPWELQETQYF (SEQ ID NO: 4), CASRYHSGQGLQETQYF (SEQ ID NO: 3), CASSLVGSGRGGTDTQYF (SEQ ID NO: 5), CASAPGGNSPLHF (SEQ ID NO: 6), CASSPGPNSPLHF (SEQ ID NO:7), CAREGDGGASNEQFF (SEQ ID NO:8), or CARGGDGRASNEQFF (SEQ ID NO: 9) and/or a TCRα comprising the sequence CAGSGAGSYQLTF (SEQ ID NO: 10), CAEGSSASKIIF (SEQ ID NO: 11), or CAMREGKGGGYNKLIF (SEQ ID NO: 12). In one aspect, disclosed herein are methods of treating, inhibiting, reducing, decreasing, ameliorating, and/or preventing a cancer and/or metastasis of any preceding aspect, wherein the TCR comprises a TCRβ comprising the sequence CASEGDGRASNEQFF (SEQ ID NO: 1) and a TCRα comprising the sequence CAGSGAGSYQLTF (SEQ ID NO: 10); a TCRβ comprising the sequence CASRYHSGQGLQETQYF (SEQ ID NO: 3) and a TCRα comprising the sequence CAMREGKGGGYNKLIF (SEQ ID NO: 12); or a TCRβ comprising the sequence CASSRPWELQETQYF (SEQ ID NO: 4) and a TCRα comprising the sequence CAEGSSASKIIF (SEQ ID NO: 11).

Also disclosed herein are methods of treating, inhibiting, reducing, decreasing, ameliorating, and/or preventing a cancer and/or metastasis of any preceding aspect, wherein the neoantigen recognized by the TCR is from a TP53^{G244C} mutant cancer (such as, for example, SSCMCGMNRR (SEQ ID NO: 32)). For example, the TCR can comprise a TCRβ comprising the sequence CASSVSGVTDTQYF (SEQ ID NO: 13), CASSLVGGYQETQYF (SEQ ID NO: 14), CASSFRTGDFSGANVLTF (SEQ ID NO: 15), CASSSPGISYEQYF (SEQ ID NO: 16), CASSFGMNTEAFF (SEQ ID NO: 17), CAISPGTYYGYTF (SEQ ID NO: 18), CASSPRDSNYGYTF (SEQ ID NO: 19), CASSVSVGVTDTQYF (SEQ ID NO: 20), CASSLTAGRALHF (SEQ ID NO: 21), or CASSTGGNSPLHF (SEQ ID NO: 22) and/or comprises a TCRα comprising the sequence CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), CAAKMNRDDKIIF (SEQ ID NO: 27), CALVGFNKFYF (SEQ ID NO: 28), CAMIPPPSVGFGNVLHC (SEQ ID NO: 29), CAVEGGGTSYGKLTF (SEQ ID NO: 30), or CAASDDSGGYQKVTF (SEQ ID NO: 31). In one aspect, disclosed herein are methods of treating, inhibiting, reducing, decreasing, ameliorating, and/or preventing a cancer and/or metastasis of any preceding aspect, wherein the TCR comprises a TCRβ comprising the sequence CASSVSGVTDTQYF (SEQ ID NO: 13) and a TCRα comprising the sequence CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), or CAAKMNRDDKIIF (SEQ ID NO: 27); a TCRβ comprising the sequence CASSLVGGYQETQYF (SEQ ID NO: 14) and a TCRα comprising the sequence CALVGFNKFYF (SEQ ID NO: 28), CAMIPPPSVGFGNVLHC (SEQ ID NO: 29); a TCRβ comprising the sequence CASSFRTGDFSGANVLTF (SEQ ID NO: 15) and a TCRα comprising the sequence CAVEGGGTSYGKLTF (SEQ ID NO: 30); or a TCRβ comprising the sequence CASSSPGISYEQYF (SEQ ID NO: 16) and a TCRα comprising the sequence CAASDDSGGYQKVTF (SEQ ID NO: 31).

Also disclosed herein are methods of treating, inhibiting, reducing, decreasing, ameliorating, and/or preventing a cancer and/or metastasis in a subject of any preceding aspect, wherein the TILs, MILs, T cells, and/or CAR T cells are expanded in vitro in the presence of one or more of the neoantigens prior to administration of the TILs.

In one aspect, disclosed herein are methods of treating, inhibiting, reducing, decreasing, ameliorating, and/or preventing a cancer and/or metastasis in a subject of any preceding aspect, further comprising administering to the subject the neoantigen which the T cell, CAR T cell, TIL, and/or MIL recognizes. In some aspects, the TILs, MILs, CAR T cell, or T cell and neoantigen are administered in the same formulation or concurrently. When administered separately, the TILs, MILs, CAR T cell, or T cells and neoantigen can be administered concurrently or 1, 2, 3, 4, 5 6, 7, 8, 9, 10, 11, 12, 13, 14, 15, 20, 25, 30, 35, 40, 45, 50, 55, 60, 65, 70, 75, 80, 85, 90, 120 min, 3, 4, 5, 6, 7, 8, 9, 10, 11, 12, 13, 14, 15, 16, 17, 18, 19, 20, 21, 22, 23, 24, 28, 30, 36, 42, 48 hours, 3, 4, 5, 6, 7, 8, 9, 10, 11, 12, 13, 14 days apart with either administration preceding the other.

Also disclosed herein are methods of treating, inhibiting, reducing, decreasing, ameliorating, and/or preventing a cancer and/or metastasis in a subject of any preceding aspect, wherein the T cells, CAR T cells, TILs, and/or MILs are obtained from the subject that is being treated.

In one aspect, disclosed herein are methods of treating, inhibiting, reducing, decreasing, ameliorating, and/or preventing a cancer and/or metastasis in a subject of any preceding aspect further comprising the administration of an anti-cancer therapeutic agent.

In one aspect, disclosed herein are methods of treating, inhibiting, reducing, decreasing, ameliorating, and/or preventing a cancer and/or metastasis in a subject of any preceding aspect further comprising administering to the subject an immune checkpoint inhibitor (such as, for example, a PD1/PDL1 blockade inhibitors and/or CTLA4/B7-1 or 2 inhibitors (such as, for example, PD-1 inhibitors lambrolizumab, OPDIVO® (Nivolumab), KEYTRUDA® (pembrolizumab), and pidilizumab; PD-L1 inhibitors BMS-936559, TECENTRIQ® (Atezolizumab), IMFINZI® (Durvalumab), and BAVENCIO® (Avelumab); and CTLA-4 inhibitors YERVOY (ipilimumab)).

Also disclosed herein are methods of treating, inhibiting, reducing, decreasing, ameliorating, and/or preventing a cancer and/or metastasis in a subject of any preceding aspect further comprising administering to the subject cancer crizotinib, ceritinib, alectinib, brigatinib, vemurafenib, dabrafenib, afatinib, Tivantinib, AMG 102, ficlatuzumab, cabozantinib, foretinib, ponatinib, onartuzumab, LKD378, AP26113, TSR-011, Selumetinib, TAE684, Trametinib, barbozatinib, gefitinib, erlotinib, paptinib, vandetanib, afatinib, osimertinib, lenvatinib, nintedanib, pazopanib, regorafenib, sorafenib, sunitinib, bosutinib, dasatinib, imatinib, nilotinib, and/or ponatinib.

Also disclosed herein are methods of identifying and/or isolating a neoantigen specific T cell comprising obtaining a neoantigen peptide of interest; mixing said peptide with labile peptide loaded FLEX-T monomers; degrading labile peptides with UV light thereby allowing for peptide exchange; mix peptide-exchanged monomers with streptavidin to produce MHC class I neoantigen specific tetramers; contacting MHC class I neoantigen specific tetramers with T cells; wherein T cells specific for the neoantigen will bind to the tetramer an can be identified by fluorescence; isolating tetramer bound T cells.

### VI. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows results from an ELISpot on TIL+DC+neoAg peptides coculture

FIG. 2 shows a results from flow cytometry on TIL+DC+neoAg peptides coculture

FIG. 3 shows a representation of an approach for identifying KRAS/TP53 neoantigen specific T cell receptors by MANAFEST assay.

FIG. 4 shows the frequency of MANAFEST+ clonotypes. Shown are 1 KRAS G12V specific TCRVB sequence clonotype CASRYHSGQGLQETQYF (SEQ ID NO: 3) and 4 TP53 G244C specific TCRVB sequence clonotypes CASSFRTGDFSGANVLTF (SEQ ID NO: 15), CASSSPGISYEQYF (SEQ ID NO: 16), CASSFGMNTEAFF (SEQ ID NO: 17), and CAISPGTYYGYTF (SEQ ID NO: 18).

FIG. 5 shows the frequency of Neoangtigen+ clonotypes. Shown are 1 KRAS G12V specific TCRVB sequence clonotype CASRYHSGQGLQETQYF (SEQ ID NO: 3) and 4 TP53 G244C specific TCRVB sequence clonotypes CASSFRTGDFSGANVLTF (SEQ ID NO: 15), CASSSPGISYEQYF (SEQ ID NO: 16), CASSFGMNTEAFF (SEQ ID NO: 17), and CAISPGTYYGYTF (SEQ ID NO: 18).

FIG. 6 shows a representation of an approach for identifying KRAS/TP53 neoantigen specific T cell receptors by isolating KRAS^{G12V} or TP53^{G244C} specific T cells using tetramer.

FIG. 7 shows that both neoantigen peptides have high peptide exchange frequency.

FIG. 8 shows flow cytometry results for KRAS^{G12V} tetramers.

FIG. 9 shows neoantigen KRAS^{G12V} specific TCRs identified by tetramer assay. Shown are the TCRβ sequences CASEGDGRASNEQFF (SEQ ID NO: 1), CASIRLRGNTDTQYF (SEQ ID NO: 2), CASRYHSGQGLQETQYF (SEQ ID NO: 3), CASSRPWELQETQYF (SEQ ID NO: 4), CASSLVGSGRGGTDTQYF (SEQ ID NO: 5), CASSVSGVTDTQYF (SEQ ID NO: 13), CASAPGGNSPLHF (SEQ ID NO:6), CASSPGPNSPLHF (SEQ ID NO:7), CAREGDGGASNEQFF (SEQ ID NO:8), and CARGGDGRASNEQFF (SEQ ID NO: 9).

FIG. 10 shows neoantigen TP53^{G244C} specific TCRs identified by tetramer assay. Shown are the TCRβ sequences CASSVSGVTDTQYF (SEQ ID NO: 13), CASSLVGSGRGGTDTQYF (SEQ ID NO: 5), CASSLVGGYQETQYF (SEQ ID NO: 14), CASAPGGNSPLHF (SEQ ID NO:6), CASSPGPNSPLHF (SEQ ID NO:7), CASIRLRGNTDTQYF (SEQ ID NO: 2), CASEGDGRASNEQFF (SEQ ID NO: 1), CASSPRDSNYGYTF (SEQ ID NO: 19), and CASSLTAGRALHF (SEQ ID NO: 21),

### VII. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

In this specification and in the claims that follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

A “binding molecule” or “antigen binding molecule” (e.g., an antibody or antigen-binding fragment thereof) as provided herein refers in its broadest sense to a molecule that specifically binds an antigenic determinant. In one embodiment, the binding molecule specifically binds to an immunoregulator molecule (such as for example, a transmembrane SEMA4D (CD100) polypeptide of about 150 kDa or a soluble SEMA4D polypeptide of about 120 kDa). In another embodiment, a binding molecule is an antibody or an antigen binding fragment thereof, e.g., MAb 67 or pepinemab.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. T CELLS COMPRISING T CELL RECEPTORS THAT ARE REACTIVE TO ONE OR MORE NEOANTIGENS AND METHODS OF THEIR USE

In one aspect, disclosed herein are methods of screening for neoantigen specific T cells, the method comprising ccccccccccccccccc

The disclosure herein provide for T cell receptors that are specific for one or more neoantigens and can be used for the treatment of a cancer. Accordingly, in one aspect, disclosed herein are T cell receptors that recognizes for one or more neoantigens including, but not limited to the TP 53^{G244C} HLA A*11:01 neoantigen SSCMCGMNRR (SEQ ID NO: 32) or the KRAS^{G12V} HLA A*11:01 neoantigen VVGAVGVGK (SEQ ID NO: 33). In one aspect, disclosed herein are T cell receptors (TCRs) that recognize one or more neoantigens (such as, for example, the TP 53^{G244C} HLA A*11:01 neoantigen SSCMCGMNRR (SEQ ID NO: 32) or the KRAS^{G12V} HLA A*11:01 neoantigen VVGAVGVGK (SEQ ID NO: 33)), wherein the TCR comprises a TCRβ comprising the sequence CASEGDGRASNEQFF (SEQ ID NO: 1), CASIRLRGNTDTQYF (SEQ ID NO: 2), CASSRPWELQETQYF (SEQ ID NO: 4), CASRYHSGQGLQETQYF (SEQ ID NO: 3), CASSLVGSGRGGTDTQYF (SEQ ID NO: 5), CASAPGGNSPLHF (SEQ ID NO:6), CASSPGPNSPLHF (SEQ ID NO:7), CAREGDGGASNEQFF (SEQ ID NO:8), CARGGDGRASNEQFF (SEQ ID NO: 9), CASSVSGVTDTQYF (SEQ ID NO: 13), CASSLVGGYQETQYF (SEQ ID NO: 14), CASSFRTGDFSGANVLTF (SEQ ID NO: 15), CASSSPGISYEQYF (SEQ ID NO: 16), CASSFGMNTEAFF (SEQ ID NO: 17), CAISPGTYYGYTF (SEQ ID NO: 18), CASSPRDSNYGYTF (SEQ ID NO: 19), CASSVSVGVTDTQYF (SEQ ID NO: 20), CASSLTAGRALHF (SEQ ID NO: 21), or CASSTGGNSPLHF (SEQ ID NO: 22). Also disclosed herein are T cell receptors, wherein the TCR further comprises a TCRα comprising the sequence CAGSGAGSYQLTF (SEQ ID NO: 10), CAEGSSASKIIF (SEQ ID NO: 11), CAMREGKGGGYNKLIF (SEQ ID NO: 12), CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), CAAKMNRDDKIIF (SEQ ID NO: 27), CALVGFNKFYF (SEQ ID NO: 28), CAMIPPPSVGFGNVLHC (SEQ ID NO: 29), CAVEGGGTSYGKLTF (SEQ ID NO: 30), or CAASDDSGGYQKVTF (SEQ ID NO: 31).

The T cell receptors disclosed herein that are specific for one or more neoantigens can be encoded for and expressed on a T cell. Thus, in one aspect, disclosed herein are T cells (such as, for example, a tumor infiltrating lymphocyte (TIL), chimeric antigen receptor (CAR) T cell, or marrow infiltrating lymphocyte (MIL)) comprising any TCR disclosed herein. The T cells encoding the TCRs disclosed herein can be engineered T cells or T cells from an autologous or allogeneic donor. In one aspect, the T cells are αβ T cells.

It is understood and herein contemplated that T cells (such as, for example, CAR T cells, MILs, and/or TILs) that are reactive to the neoantigens disclosed herein can be administered to a subject with a cancer as a treatment for said cancer. Thus, once identified, the neoantigens can be used to screen for T cells comprising TCRs reactive to the neoantigen and once identified, said T cells can be expanded (in the presence of the neoantigens) and administered to a patient with a cancer. Accordingly, in one aspect, disclosed herein are methods of screening for T cells reactive to a neoantigen, the method comprising obtaining a neoantigen peptide of interest; mixing said peptide with labile peptide loaded FLEX-T monomers; degrading labile peptides with UV light thereby allowing for peptide exchange; mix peptide-exchanged monomers with streptavidin to produce MHC class I neoantigen specific tetramers; contacting MHC class I neoantigen specific tetramers with T cells; wherein T cells specific for the neoantigen bind to the tetramer an can be identified by fluorescence. The tetramer bound T cells can then be isolated using a cell sorting technique such as FACS.

Because the T cells (such as of T cells), TILs, MILs, and/or CAR T cells that bind the neotantigen, the neoantigen can be used to enrich neoantigen specific T cells (such as αβ T cells), TILs, MILs, and/or CAR T cells from a mixed population of T cells. This can be done by co-culturing the neoantigen with the T cells (such as αβ T cells), TILs, MILs, and/or CAR T cells as the T cells (such as of T cells), TILs, MILs, and/or CAR T cells specific for the neoantigen will expand and thus comprise a larger portion of the mixed T cell population following culture. Alternatively, the neoantigens can be made to adhere to device or conjugated to a magnetic bead, column, or antibody and neoantigen specific T cells (such as αβ T cells), TILs, MILs, and/or CAR T cells can be bound to the beads, columns, or antibodies and thus separated by non-specific T cells.

In one aspect, it is understood that neoantigens made be administered directly t a subject with a cancer to generate neoantigen reactive T cells, TILs, or MILs in vivo. However, as noted above, the T cells, TILs, MILs, and CAR T cells (such as αβ T cells, TILs, MILS, and CAR T cells comprising TCR reactive to the neoantigens) disclosed herein can be effective in the treatment, inhibition, reduction, amelioration and/or prevention of a cancer. Thus, disclosed herein are methods of treating, inhibiting, reducing, decreasing, ameliorating, and/or preventing a cancer and/or metastasis (such as, for example, a tyrosine kinase inhibitor resistant or EGFR mutated cancer) in a subject comprising administering to the subject any TCRs, T cells, CAR T cells, TILs, and/or MILs disclosed herein that is reactive to one or more neoantigens. For example disclosed herein are methods of treating, inhibiting, reducing, decreasing, ameliorating, and/or preventing a cancer and/or metastasis (such as, for example, a tyrosine kinase inhibitor resistant or EGFR mutated cancer) in a subject comprising administering to the subject a T cell, CAR T cell, TIL, and/or MIL comprising a TCR that recognizes a neoantigen including, but not limited to, neoantigens comprising the TP53^{G244C} sequence SSCMCGMNRR (SEQ ID NO: 32) or the KRAS^{G12V} HLA A*11:01 neoantigen VVGAVGVGK (SEQ ID NO: 33).

Accordingly, in one aspect, disclosed herein are methods of treating, inhibiting, reducing, decreasing, ameliorating, and/or preventing a cancer and/or metastasis in a subject comprising administering to the subject any of the TCRs, T cells, CAR T cells, TILs, and/or MILs disclosed herein. For example, disclosed herein are methods of treating a cancer in a subject comprising administering to the subject a T cell, CAR T cell, TIL, and/or MIL comprising a TCR that recognizes a neoantigen including, but not limited to, neoantigens comprising the TP53^{G244C} sequence SSCMCGMNRR (SEQ ID NO: 32) or the KRAS^{G12V} HLA A*11:01 neoantigen VVGAVGVGK (SEQ ID NO: 33). For example, disclosed herein are methods of treating, inhibiting, reducing, decreasing, ameliorating, and/or preventing a cancer and/or metastasis in a subject comprising administering to the subject any of the TCRs, T cells, CAR T cells, TILs, and/or MILs disclosed herein. For example disclosed herein are methods of treating a cancer in a subject comprising administering to the subject a T cell, CAR T cell, TIL, and/or MIL comprising a TCR a TCRβ comprising the sequence CASEGDGRASNEQFF (SEQ ID NO: 1), CASIRLRGNTDTQYF (SEQ ID NO: 2), CASSRPWELQETQYF (SEQ ID NO: 4), CASRYHSGQGLQETQYF (SEQ ID NO: 3), CASSLVGSGRGGTDTQYF (SEQ ID NO: 5), CASAPGGNSPLHF (SEQ ID NO:6), CASSPGPNSPLHF (SEQ ID NO:7), CAREGDGGASNEQFF (SEQ ID NO:8), CARGGDGRASNEQFF (SEQ ID NO: 9), CASSVSGVTDTQYF (SEQ ID NO: 13), CASSLVGGYQETQYF (SEQ ID NO: 14), CASSFRTGDFSGANVLTF (SEQ ID NO: 15), CASSSPGISYEQYF (SEQ ID NO: 16), CASSFGMNTEAFF (SEQ ID NO: 17), CAISPGTYYGYTF (SEQ ID NO: 18), CASSPRDSNYGYTF (SEQ ID NO: 19), CASSVSVGVTDTQYF (SEQ ID NO: 20), CASSLTAGRALHF (SEQ ID NO: 21), or CASSTGGNSPLHF (SEQ ID NO: 22) and/or a TCRα comprising the sequence CAGSGAGSYQLTF (SEQ ID NO: 10), CAEGSSASKIIF (SEQ ID NO: 11), CAMREGKGGGYNKLIF (SEQ ID NO: 12), CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), CAAKMNRDDKIIF (SEQ ID NO: 27), CALVGFNKFYF (SEQ ID NO: 28), CAMIPPPSVGFGNVLHC (SEQ ID NO: 29), CAVEGGGTSYGKLTF (SEQ ID NO: 30), or CAASDDSGGYQKVTF (SEQ ID NO: 31).

In one aspect, disclosed herein are methods of treating, inhibiting, reducing, decreasing, ameliorating, and/or preventing a cancer and/or metastasis, wherein the neoantigen recognized by the TCR is from a KRAS^{G12V} mutant cancer (such as, for example, VVGAVGVGK (SEQ ID NO: 33)). For example, the TCR can comprise a TCRβ comprising the sequence CASEGDGRASNEQFF (SEQ ID NO: 1), CASIRLRGNTDTQYF (SEQ ID NO: 2), CASSRPWELQETQYF (SEQ ID NO: 4), CASRYHSGQGLQETQYF (SEQ ID NO: 3), CASSLVGSGRGGTDTQYF (SEQ ID NO: 5), CASAPGGNSPLHF (SEQ ID NO:6), CASSPGPNSPLHF (SEQ ID NO:7), CAREGDGGASNEQFF (SEQ ID NO:8), or CARGGDGRASNEQFF (SEQ ID NO: 9) and/or a TCRα comprising the sequence CAGSGAGSYQLTF (SEQ ID NO: 10), CAEGSSASKIIF (SEQ ID NO: 11), or CAMREGKGGGYNKLIF (SEQ ID NO: 12). In one aspect, disclosed herein are methods of treating, inhibiting, reducing, decreasing, ameliorating, and/or preventing a cancer and/or metastasis, wherein the TCR comprises a TCRβ comprising the sequence CASEGDGRASNEQFF (SEQ ID NO: 1) and a TCRα comprising the sequence CAGSGAGSYQLTF (SEQ ID NO: 10); a TCRβ comprising the sequence CASRYHSGQGLQETQYF (SEQ ID NO: 3) and a TCRα comprising the sequence CAMREGKGGGYNKLIF (SEQ ID NO: 12); or a TCRβ comprising the sequence CASSRPWELQETQYF (SEQ ID NO: 4) and a TCRα comprising the sequence CAEGSSASKIIF (SEQ ID NO: 11).

Also disclosed herein are methods of treating, inhibiting, reducing, decreasing, ameliorating, and/or preventing a cancer and/or metastasis, wherein the neoantigen recognized by the TCR is from a TP53^{G244C} mutant cancer (such as, for example, SSCMCGMNRR (SEQ ID NO: 32)). For example, the TCR can comprise a TCRβ comprising the sequence CASSVSGVTDTQYF (SEQ ID NO: 13), CASSLVGGYQETQYF (SEQ ID NO: 14), CASSFRTGDFSGANVLTF (SEQ ID NO: 15), CASSSPGISYEQYF (SEQ ID NO: 16), CASSFGMNTEAFF (SEQ ID NO: 17), CAISPGTYYGYTF (SEQ ID NO: 18), CASSPRDSNYGYTF (SEQ ID NO: 19), CASSVSVGVTDTQYF (SEQ ID NO: 20), CASSLTAGRALHF (SEQ ID NO: 21), or CASSTGGNSPLHF (SEQ ID NO: 22) and/or comprises a TCRα comprising the sequence CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), CAAKMNRDDKIIF (SEQ ID NO: 27), CALVGFNKFYF (SEQ ID NO: 28), CAMIPPPSVGFGNVLHC (SEQ ID NO: 29), CAVEGGGTSYGKLTF (SEQ ID NO: 30), or CAASDDSGGYQKVTF (SEQ ID NO: 31). In one aspect, disclosed herein are methods of treating, inhibiting, reducing, decreasing, ameliorating, and/or preventing a cancer and/or metastasis, wherein the TCR comprises a TCRβ comprising the sequence CASSVSGVTDTQYF (SEQ ID NO: 13) and a TCRα comprising the sequence CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), or CAAKMNRDDKIIF (SEQ ID NO: 27); a TCRβ comprising the sequence CASSLVGGYQETQYF (SEQ ID NO: 14) and a TCRα comprising the sequence CALVGFNKFYF (SEQ ID NO: 28), CAMIPPPSVGFGNVLHC (SEQ ID NO: 29); a TCRβ comprising the sequence CASSFRTGDFSGANVLTF (SEQ ID NO: 15) and a TCRα comprising the sequence CAVEGGGTSYGKLTF (SEQ ID NO: 30); or a TCRβ comprising the sequence CASSSPGISYEQYF (SEQ ID NO: 16) and a TCRα comprising the sequence CAASDDSGGYQKVTF (SEQ ID NO: 31).

It is understood and herein contemplated that the disclosed cancer treatment, inhibition, reduction, amelioration and/or prevention methods can use T cells obtained directly from a subject receiving T cell immunotherapy (i.e., an autologous donor) or from another source (for example an allogeneic donor or engineered T cell). In one aspect, the methods can further comprise obtaining said T cells. Thus, also disclosed herein are methods of treating, inhibiting, reducing, decreasing, ameliorating, and/or preventing a cancer and/or metastasis further comprising obtaining peripheral blood mononuclear cells (PBMCs) from the subject with the cancer or allogeneic donor. T cells can be isolated from the PBMC from the subject using cell sorting techniques known in the art, including but not limited to magnetic cell sorting (MACS) or fluorescence acquired cell sorting (FACS).

In one aspect, disclosed herein are methods of treating, inhibiting, reducing, decreasing, ameliorating, and/or preventing a cancer and/or metastasis wherein the isolated T cells are co-cultured with the putative neoantigens of step e and assayed for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

Also disclosed herein are methods of treating, inhibiting, reducing, decreasing, ameliorating, and/or preventing a cancer and/or metastasis in a subject, wherein the TILs, MILs, T cells, and/or CAR T cells are expanded in vitro in the presence of one or more of the neoantigens prior to administration of the TILs.

In one aspect, the methods of treating, inhibiting, reducing, decreasing, ameliorating, and/or preventing a cancer and/or metastasis can further comprise administering neoantigen reactive T cells (including, but not limited to, TILs, MILs, and CAR T cells) in combination with any of the disclosed neoantigens or any neoantigen for which the T cell receptor of the T cell is specific. It is understood and herein contemplated that the neoantigens and T cells (including, but not limited to, TILs, MILs, and CAR T cells) can be administered in the same formulation, or separately. When administered separately, the T cells (including, but not limited to, TILs, MILs, and CAR T cells) and neoantigen can be administered concurrently or 1, 2, 3, 4, 5 6, 7, 8, 9, 10, 11, 12, 13, 14, 15, 20, 25, 30, 35, 40, 45, 50, 55, 60, 65, 70, 75, 80, 85, 90, 120 min, 3, 4, 5, 6, 7, 8, 9, 10, 11, 12, 13, 14, 15, 16, 17, 18, 19, 20, 21, 22, 23, 24, 28, 30, 36, 42, 48 hours, 3, 4, 5, 6, 7, 8, 9, 10, 11, 12, 13, 14 days apart with either administration preceding the other. In some aspects, the methods can comprise lymphodepletion prior to administration of the disclosed T cells (such as, for example αβ T cells), TILs, MILs, and/or CAR T cells. Additionally, IL-2 can be administered to the subject following infusion of the T cells (such as, for example αβ T cells), TILs, MILs, and/or CAR T cells.

The disclosed methods and any neoantigen disclosed herein can be used to treat, inhibit, reduce, decrease, ameliorate, and/or prevent any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, non-small cell lung cancer (NSCLC), or pancreatic cancer. In particular, it is understood and herein contemplated that the cancer can be a TP53 mutated cancer or KRAS mutated cancer.

The Kirsten rat sarcoma viral oncogene homolog (KRAS) gene is one of the first described oncogenes. KRAS mutations are present in approximately 30% of lung adenocarcinomas and less commonly in squamous NSCLC (~5%). They are found more frequently in Caucasians with lung cancer than in the Asian population and in current- or ex-smokers when compared with never-smokers. Most KRAS mutations in NSCLC are single amino acid substitutions in codon 12 (80%) and to a lesser extent in codons 13 and 61. In current- or ex-smokers, KRAS mutations are usually transversions (pyrimidine nucleotide is exchanged for purine or vice versa; e.g., G→T or G→C) and transitions in never-smokers (purine nucleotide is exchanged for another purine or pyrimidine for another pyrimidine; e.g., G→A or C→T). KRAS mutations are nearly always mutually exclusive with EGFR and BRAF mutations although rare co-existence of EGFR and KRAS mutations has been observed. KRAS mutations co-exist with PIK3CA mutations in approximately 19% of PIK3CA-mutant NSCLC.

It has been postulated for over 20 years that KRAS-mutant NSCLC may be associated with poor outcome. However, multiple studies have shown conflicting results due to heterogeneity among the studies, including tumor type, stage, treatment, and study end points. A meta-analysis of 28 studies published in 2005 demonstrated that KRAS mutation was a significant prognostic marker when polymerase chain reaction sequencing was used as a detection method. Recently published results of a LACE-Bio pooled retrospective analysis reported no prognostic or predictive (in regard to benefit from adjuvant chemotherapy) effect of KRAS mutations in patients with resected NSCLC. A subset analysis of patients with NSCLC with KRAS codon 13 mutations suggests that adjuvant chemotherapy may have a deleterious effect in this subgroup, but needs to be further validated (HR-5.78; 95% CI, 2.06-16.2). In the absence of prospective, large, randomized clinical trials, KRAS mutation status in NSCLC cannot be used as a prognostic nor predictive biomarker for treatment with exception of negative predictive value of KRAS mutations.

Direct inhibition of KRAS has been unsuccessful so far due to its molecular and functional complexity. The activation of the RAS-RAF-MEK-ERK signaling pathway as a consequence of KRAS mutations renders it an attractive target for small-molecule inhibition in KRAS-mutated NSCLC. Given the critical location in this signaling pathway, MEK has been recognized as an important target, downstream from KRAS, for anti-cancer therapy.

KRAS mutations in NSCLC, despite being the most common, remain the most intriguing and elusive of therapeutic targets. At present, targeted treatment is not available for KRAS-mutated NSCLC outside clinical trials. However, novel agents targeting downstream effector signaling pathways are under clinical development.

In one aspect, it is understood the treatment of cancer does not need to be limited to the administration of neoantigens and/or neoantigen-specific T cells, but can include the further administration of anti-cancer agents to treat, inhibit, reduce, decrease, ameliorate, and/or prevent a cancer and/or metastasis. Anti-cancer therapeutic agents (such as chemotherapeutics, immunotoxins, peptides, and antibodies) that can be used in the methods of treating, inhibiting, reducing, decreasing, ameliorating, and/or preventing a cancer and/or metastasis and in combination with any of the disclosed neoantigens or any CAR T cells, TIL, or MIL specific for said neoantigen can comprise any anti-cancer therapeutic agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

In one aspect, it is understood that the additional anti-cancer agent administered in addition to the any TCRs, T cells, CAR T cells, TILs, and/or MILs disclosed herein for the treatment, decrease, reduction, inhibition, amelioration, and/or prevention of a cancer or metastasis can be an immune checkpoint inhibitor. Thus, in one aspect, disclosed herein are methods of treating, inhibiting, reducing, decreasing, ameliorating, and/or preventing a cancer and/or metastasis in a subject further comprising administering to the subject an immune checkpoint inhibitor, wherein the checkpoint inhibitor comprises a PD1/PDL1 blockade inhibitors and/or CTLA4/B7-1 or 2 inhibitors (such as, for example, PD-1 inhibitors lambrolizumab, OPDIVO® (Nivolumab), KEYTRUDA® (pembrolizumab), and pidilizumab; PD-L1 inhibitors BMS-936559, TECENTRIQ® (Atezolizumab), IMFINZI® (Durvalumab), and BAVENCIO® (Avelumab); and CTLA-4 inhibitors YERVOY (ipilimumab).

As noted above, the cancers to be treated, inhibited, reduced, decreased, ameliorated, and/or prevented by the methods disclosed herein can comprise a tyrosine kinase inhibitor resistant cancer. It is understood and herein contemplated that administration of the disclosed TCRs, T cells, CAR T cells, TILs, and/or MILs can make a cancer that is resistant to a tyrosine kinase inhibitor more susceptible to said treatment. Thus, in one aspect, disclosed herein are methods of treating, inhibiting, reducing, decreasing, ameliorating, and/or preventing a cancer and/or metastasis in a subject further comprising administering to the subject crizotinib, ceritinib, alectinib, brigatinib, vemurafenib, dabrafenib, afatinib, Tivantinib, AMG 102, ficlatuzumab, cabozantinib, foretinib, ponatinib, onartuzumab, LKD378, AP26113, TSR-011, Selumetinib, TAE684, Trametinib, barbozatinib, gefitinib, erlotinib, paptinib, vandetanib, afatinib, osimertinib, lenvatinib, nintedanib, pazopanib, regorafenib, sorafenib, sunitinib, bosutinib, dasatinib, imatinib, nilotinib, and/or ponatinib.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### Example 1

This process is developed to identify neoantigens and then utilize neoantigens to isolate neoantigen specific TILs for the treatment of a cancer. PBMCs and/or tissue resections were collected from patients before and after combination therapy. PBMC were subjected to magnetic cell separation (MACS) to isolate T cells from the PBMC. Concurrently, metastatic tissue resections were either fragmented or subjected to sequence analysis through whole exosome sequencing (WES) or RNAseq analysis. The fragmented sections were cultured to expand tumor infiltrating lymphocytes. Bioinformatic analysis was performed on the sequencing data for the determination of the number and sequence of each of the productive unique VB gene identified within each sample and the degree of clone sharing between samples.

To assay the reactivity of the unique peptides TCRVβ sequencing was used. Briefly, potential neoantigens were contacted with TILs isolated from either expanded from fragmented tissue resections or isolated via MACS from PBMC and the TVRß sequencing performed to show reactivity.

To define the immunogenicity of every individual candidate predicted in the analysis of mutational burden analysis, the presence of reactive T cell clones in peripheral blood were individually quantified in MANAFEST analysis. MANAFEST is a novel, scalable method to evaluate candidate tumor neoantigens for their ability to induce T cell responses (more sensitive and specific than conventional ELISPOT or ELISA). Briefly, up to 30 mutations associated neoantigen candidates per tumor (MANAs) were synthesized (New England Peptide, Inc) for reactivity analysis. The T cell fraction of PBMCs collected before and 3 and 7 weeks after initial treatment for every patient were separated using beads. The non-T cell fraction is then gamma-irradiated and autologous T and irradiated non-T cells are put back together. Predicted neoantigens are then added to specific wells in triplicate, along with cytokines (such as, IL-2, IL-7 and IL-15). On days 3 and 7, half the medium is replaced with fresh medium containing cytokines. CD8 T cells are then separated using a CD4 positive selection kit and subjected to ImmunoSeq analysis. Controls include CEF peptides (epitopes from common viral infections) or no peptide. TCR beta chains expanded >10-fold in response to individual peptides (but not in response to other neoantigens or control CEF peptides) are considered positive. The MANAFEST+ data for various clonotypes reveal that Neoantigen specific clones exist in some of TIL fragments as well as postREP. Additionally, the frequencies of neoantigens increased dramatically after TIL infusion and were very low in baseline tumors.

In a similar manner it was postulated that MHC class I tetramer technology can be used to identify and isolate neoantigen specific T cells. Briefly, a neoantigen peptide of interest is obtained and mixed with labile peptide loaded FLEX-T monomers; The labile peptides with UV light thereby allowing for substitutions by the peptide of interest (i.e., peptide exchange). Next peptide-exchanged monomers are mixed with streptavidin to produce MHC class I neoantigen specific tetramers. The neoantigen specific tetramers can then be mixed with putative neoantigen specific T cells; the T cells binding the MHC class I tetramers being the neoantigen specific T cells which then can be separated from non-specific T cells by FACS.

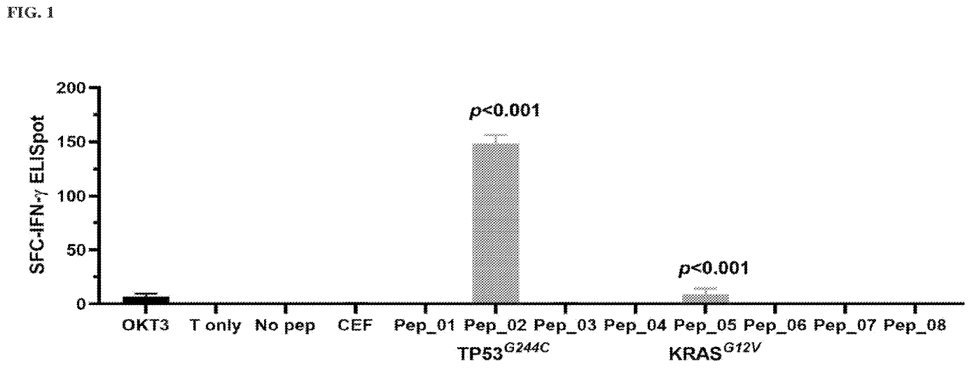

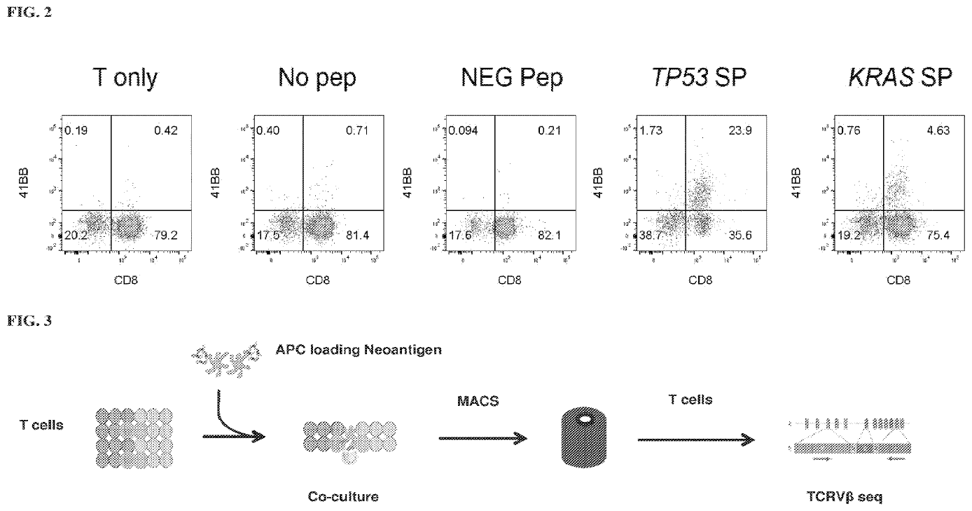

To investigate the ability to isolate neoantigen specific T cells by MHC class tetramers, we obtained HLA-A*11:01 peptides from TP53 G244C mutated and KRAS G12V mutated cancers. The TP53 G244C mutation is present in 0.07% of AACR GENIE cases, with lung adenocarcinoma and colon adenocarcinoma having the greatest prevalence. ELISpot (FIG. 1) and Flow cytometry (FIG. 2) revealed the presence of neoantigens that were prevalent in these mutated cancers. As shown in Table 1, the HLA A*11:01 neoantigen peptide for these TP53 mutated cancers is SSCMCGMNRR (SEQ ID NO: 32). Similarly, KRAS G12V is present in 2.95% of AACR GENIE cases, with pancreatic adenocarcinoma, lung adenocarcinoma, colon adenocarcinoma, colorectal adenocarcinoma, and rectal adenocarcinoma having the greatest prevalence. As shown in Table 1, the HLA A*11:01 neoantigen peptide for these KRAS mutated cancers is VVGAVGVGK (SEQ ID NO: 33).

TABLE 1
netMHCpan prediction

| | | | affinity_ | affinity_ |
|---|---|---|---|---|
| HLA | symbol | peptide_mut | ref(nM) | mut(nM) |
| HLA-A*11:01 | TP53 | SSCMCGMNRR | 114.2 | 138.1 |
| | | (SEQ ID NO: 32) | | |
| HLA-A*11:01 | KRAS | VVGAVGVGK | 139.3 | 66.9 |
| | | (SEQ ID NO: 33) | | |

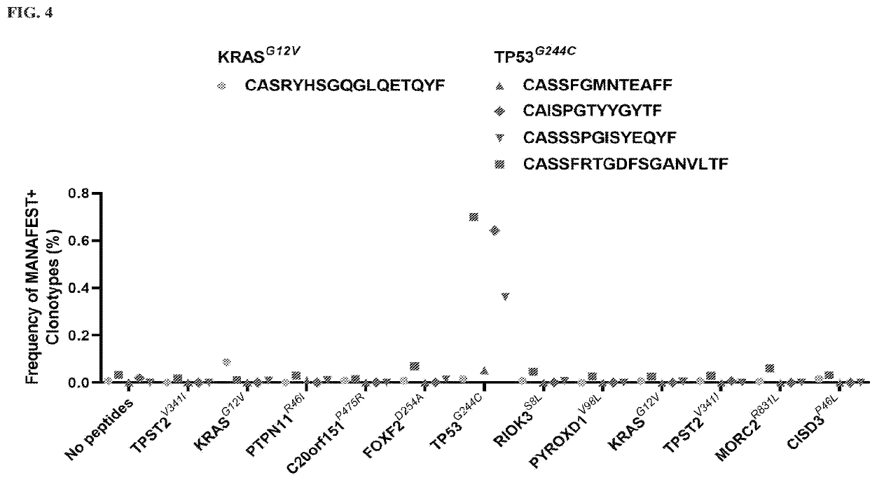

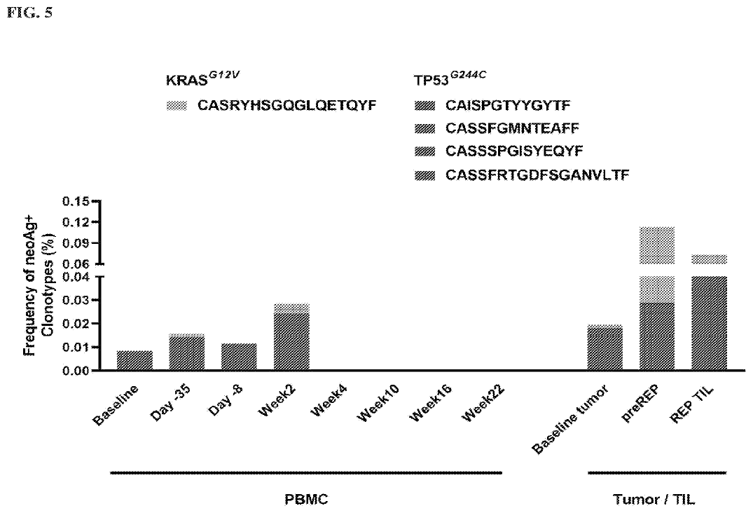

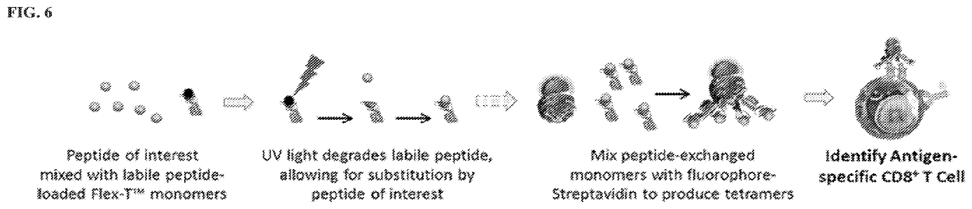

Using the MANAFEST assay (FIG. 3) we were able to identify 1 KRAS G12V specific TCRVβ sequence clonotype CASRYHSGQGLQETQYF (SEQ ID NO: 3) and 4 TP53 G244C specific TCRVβ sequence clonotypes CASSFRTGDFSGANVLTF (SEQ ID NO: 15), CASSSPGISYEQYF (SEQ ID NO: 16), CASSFGMNTEAFF (SEQ ID NO: 17), and CAISPGTYYGYTF (SEQ ID NO: 18) (see FIGS. 4 and 5).

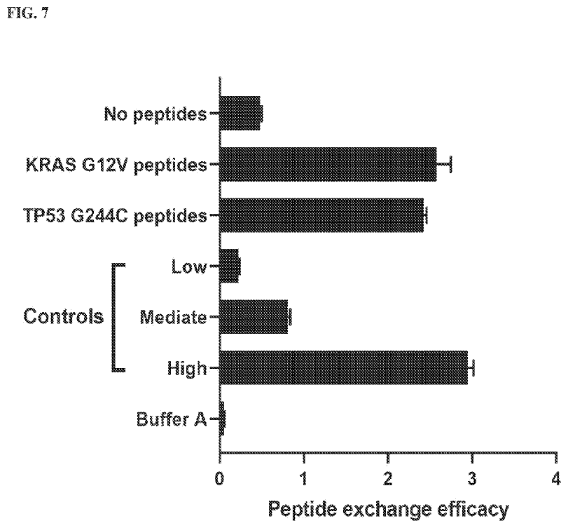

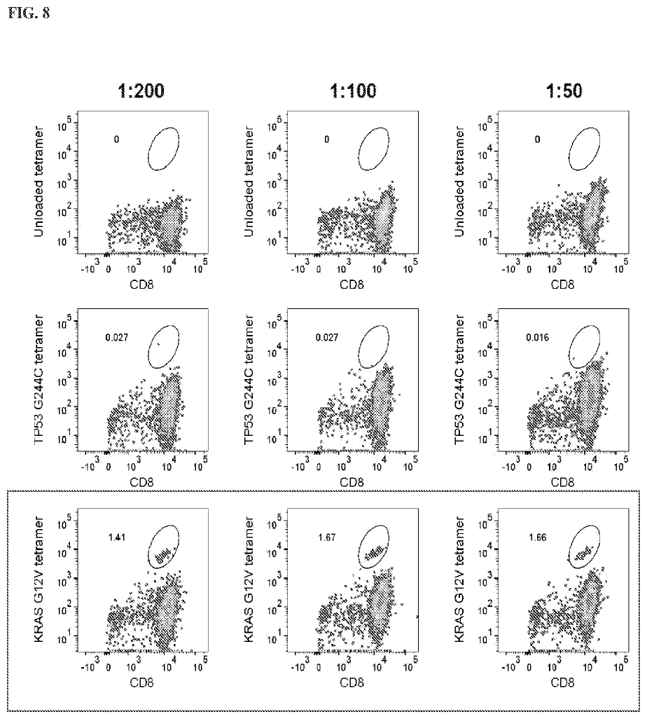

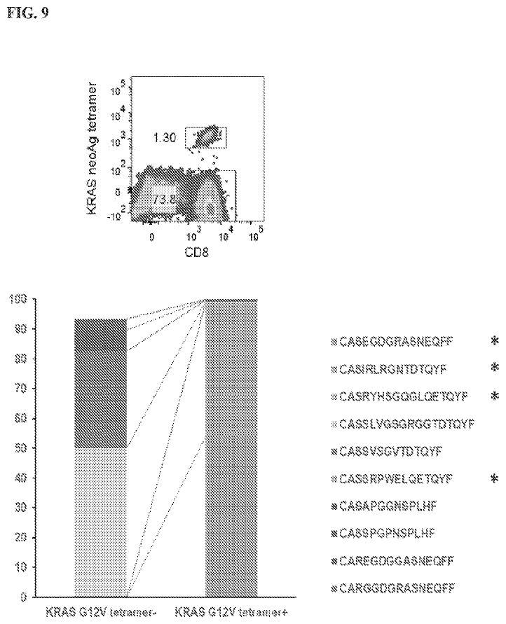

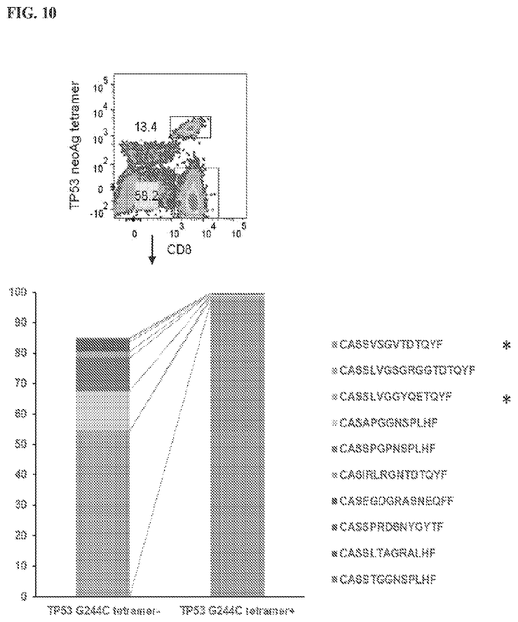

By applying the MHC class I tetramer approach (FIGS. 6-8). KRAS^{G12V} and TP53^{G244C} neoantigen epitopes were used for HLA-A11*01 tetramer construction. TILs stained by the tetramer were sorted and their TCRs were sequenced. Frequencies of TCRs between tetramer positive and negative populations were compared and those highly enriched in tetramer positive populations (* labeled) were identified to be neoantigen specific TCRs (FIGS. 9 and 10). As seen in Table 2, Three more KRAS G12V specific TCRs identified from KRAS G12V tetramer+ sorted TILs (3/4 have TRA identified, ID 4 has been identified by MANAFEST assay and has been submitted in our last patent. Therefore, I listed 4 for KRAS G12V, but only three (ID1-3) are newly added in this patent.). Two more TP53 G244C specific TCRs identified from tetramer+ sorted TILs, shown in ID5-6; ID7-8 were identified by MANAFEST and have been submitted in our last patent, but now we have the paired TRA sequences.

TABLE 2

| | | | | Approach |
|---|---|---|---|---|
| ID | TARGET | TRB | TRA | Applied |
| 1 | KRAS | CASEGDGRASNEQFF | CAGSGAGSYQLTF | tetramer |
| | G12V | (SEQ ID NO: 1) | (SEQ ID NO: 10) | |
| 2 | KRAS | CASIRLRGNTDTQYF | | tetramer |
| | G12V | (SEQ ID NO: 2) | | |
| 3 | KRAS | CASSRPWELQETQYF | CAEGSSASKIIF | tetramer |
| | G12V | (SEQ ID NO: 4) | (SEQ ID NO: 11) | |
| 4 | KRAS | CASRYHSGQGLQETQYF | CAMREGKGGGYNKLIF | Tetramer & |
| | G12V | (SEQ ID NO: 3) | (SEQ ID NO: 12) | MANAFEST |
| 5 | TP53 | CASSVSGVTDTQYF | CAENDSYGGATNKLIF | tetramer |
| | G244C | (SEQ ID NO: 13) | (SEQ ID NO: 23) | |
| | | | CAMSTAGGTSYGKLTF | |
| | | | (SEQ ID NO: 24) | |
| | | | CAVLIQGAQKLVF | |
| | | | (SEQ ID NO: 25) | |
| | | | CAYRTLHSGNTPLVF | |
| | | | (SEQ ID NO: 26) | |
| | | | CAAKMNRDDKIIF | |
| | | | (SEQ ID NO: 27) | |
| 6 | TP53 | CASSLVGGYQETQYF | CALVGFNKFYF | tetramer |
| | G244C | (SEQ ID NO: 14) | (SEQ ID NO: 28) | |
| | | | CAMIPPPSVGFGNVLHC | |
| | | | (SEQ ID NO: 29) | |
| 7 | TP53 | CASSFRTGDFSGANVLTF | CAVEGGGTSYGKLTF | MANAFEST |
| | G244C | (SEQ ID NO: 15) | (SEQ ID NO: 30) | |
| 8 | TP53 | CASSSPGISYEQYF | CAASDDSGGYQKVTF | MANAFEST |
| | G244C | (SEQ ID NO: 16) | (SEQ ID NO: 31) | |

1. (canceled)

2. A T cell receptor (TCR) that recognizes one or more neoantigens, wherein the TCR comprises a TCRβ comprising the sequence CASEGDGRASNEQFF (SEQ ID NO: 1), CASIRLRGNTDTQYF (SEQ ID NO: 2), CASSRPWELQETQYF (SEQ ID NO: 4), CASRYHSGQGLQETQYF (SEQ ID NO: 3), CASSLVGSGRGGTDTQYF (SEQ ID NO: 5), CASAPGGNSPLHF (SEQ ID NO:6), CASSPGPNSPLHF (SEQ ID NO:7), CAREGDGGASNEQFF (SEQ ID NO:8), CARGGDGRASNEQFF (SEQ ID NO: 9), CASSVSGVTDTQYF (SEQ ID NO: 13), CASSLVGGYQETQYF (SEQ ID NO: 14), CASSFRTGDFSGANVLTF (SEQ ID NO: 15), CASSSPGISYEQYF (SEQ ID NO: 16), CASSFGMNTEAFF (SEQ ID NO: 17), CAISPGTYYGYTF (SEQ ID NO: 18), CASSPRDSNYGYTF (SEQ ID NO: 19), CASSVSVGVTDTQYF (SEQ ID NO: 20), CASSLTAGRALHF (SEQ ID NO: 21), or CASSTGGNSPLHF (SEQ ID NO: 22).

3. The TCR of claim 2, wherein the TCR further comprises a TCRα comprising the sequence CAGSGAGSYQLTF (SEQ ID NO: 10), CAEGSSASKIIF (SEQ ID NO: 11), CAMREGKGGGYNKLIF (SEQ ID NO: 12), CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), CAAKMNRDDKIIF (SEQ ID NO: 27), CALVGFNKFYF (SEQ ID NO: 28), CAMIPPPSVGFGNVLHC (SEQ ID NO: 29), CAVEGGGTSYGKLTF (SEQ ID NO: 30), or CAASDDSGGYQKVTF (SEQ ID NO: 31).

4. A T cell comprising the TCR of claim 2.

5. The T cell of claim 4, wherein the T cell is a tumor infiltrating lymphocyte (TIL), chimeric antigen receptor (CAR) T cell, or marrow infiltrating lymphocyte (MIL).

6. A method of treating a cancer in a subject comprising administering to the subject a T cell, CAR T cell, TIL, and/or MIL of claim 5.

7. A method of treating a cancer in a subject comprising administering to the subject a T cell, CAR T cell, TIL, and/or MIL comprising a TCR that recognizes one or more neoantigens.

8. The method of claim 7, wherein the one or more neoantigens recognized by the TCR is derived from a KRAS G12V mutant cancer.

9. The method of claim 8, wherein the TCR comprises a TCRβ comprising the sequence CASEGDGRASNEQFF (SEQ ID NO: 1), CASIRLRGNTDTQYF (SEQ ID NO: 2), CASSRPWELQETQYF (SEQ ID NO: 4), CASRYHSGQGLQETQYF (SEQ ID NO: 3), CASSLVGSGRGGTDTQYF (SEQ ID NO: 5), CASAPGGNSPLHF (SEQ ID NO:6), CASSPGPNSPLHF (SEQ ID NO:7), CAREGDGGASNEQFF (SEQ ID NO:8), or CARGGDGRASNEQFF (SEQ ID NO: 9).

10. The method of claim 9, wherein the TCR further comprises a TCRα comprising the sequence CAGSGAGSYQLTF (SEQ ID NO: 10), CAEGSSASKIIF (SEQ ID NO: 11), or CAMREGKGGGYNKLIF (SEQ ID NO: 12).

11. The method of claim 10, wherein the TCR comprises:
(i) a TCRβ comprising the sequence CASEGDGRASNEQFF (SEQ ID NO: 1) and a TCRα comprising the sequence CAGSGAGSYQLTF (SEQ ID NO: 10);
(ii) a TCRβ comprising the sequence CASRYHSGQGLQETQYF (SEQ ID NO: 3) and a TCRα comprising the sequence CAMREGKGGGYNKLIF (SEQ ID NO: 12); or
(iii) a TCRβ comprising the sequence CASSRPWELQETQYF (SEQ ID NO: 4) and a TCRα comprising the sequence CAEGSSASKIIF (SEQ ID NO: 11).

12. The method of claim 7, wherein the one or more neoantigens recognized by the TCR is derived from a TP53 G244C mutant cancer.

13. The method of claim 8, wherein the TCR comprises a TCRβ comprising the sequence CASSVSGVTDTQYF (SEQ ID NO: 13), CASSLVGGYQETQYF (SEQ ID NO: 14), CASSFRTGDFSGANVLTF (SEQ ID NO: 15), CASSSPGISYEQYF (SEQ ID NO: 16), CASSFGMNTEAFF (SEQ ID NO: 17), CAISPGTYYGYTF (SEQ ID NO: 18), CASSPRDSNYGYTF (SEQ ID NO: 19), CASSVSVGVTDTQYF (SEQ ID NO: 20), CASSLTAGRALHF (SEQ ID NO: 21), or CASSTGGNSPLHF (SEQ ID NO: 22).

14. The method of claim 9, wherein the TCR further comprises a TCRα comprising the sequence CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), CAAKMNRDDKIIF (SEQ ID NO: 27), CALVGFNKFYF (SEQ ID NO: 28), CAMIPPPSVGFGNVLHC (SEQ ID NO: 29), CAVEGGGTSYGKLTF (SEQ ID NO: 30), or CAASDDSGGYQKVTF (SEQ ID NO: 31).

15. The method of claim 12, wherein the TCR comprises:
(i) a TCRβ comprising the sequence CASSVSGVTDTQYF (SEQ ID NO: 13) and a TCRα comprising the sequence CAENDSYGGATNKLIF (SEQ ID NO: 23), CAMSTAGGTSYGKLTF (SEQ ID NO: 24), CAVLIQGAQKLVF (SEQ ID NO: 25), CAYRTLHSGNTPLVF (SEQ ID NO: 26), or CAAKMNRDDKIIF (SEQ ID NO: 27);
(ii) a TCRβ comprising the sequence CASSLVGGYQETQYF (SEQ ID NO: 14) and a TCRα comprising the sequence CALVGFNKFYF (SEQ ID NO: 28), CAMIPPPSVGFGNVLHC (SEQ ID NO: 29);
(iii)_a TCRβ comprising the sequence CASSFRTGDFSGANVLTF (SEQ ID NO: 15) and a TCRα comprising the sequence CAVEGGGTSYGKLTF (SEQ ID NO: 30); or
(iv) a TCRβ comprising the sequence CASSSPGISYEQYF (SEQ ID NO: 16) and a TCRα comprising the sequence CAASDDSGGYQKVTF (SEQ ID NO: 31).

16. The method of claim 7, wherein the TILs, MILs, T cells, and/or CAR T cells are expanded in vitro in the presence of one or more of the neoantigens prior to administration of the TILs.

17. The method of claim 7, further comprising administering to the subject the one or more neoantigens which the T cell, CAR T cell, TIL, and/or MIL recognizes.

18. The method of claim 17, wherein the TILs and one or more neoantigens are administered in the same formulation.

19. The method of claim 17, wherein the TILs and one or more neoantigens are administered concurrently.

20. The method of claim 7, wherein the T cells, CAR T cells, TILs, and/or MILs are obtained from the subject that is being treated.

21. The method of claim 7 further comprising administering to the subject an immune checkpoint inhibitor.

22. The method of claim 21, wherein the checkpoint inhibitor comprises;
(i) a PD-1 inhibitor selected from the group consisting of inhibitors lambrolizumab, OPDIVO® (Nivolumab), KEYTRUDA® (pembrolizumab), and pidilizumab;
(ii) a PD-L1 inhibitor selected from the group consisting of BMS-936559, TECENTRIQ® (Atezolizumab), IMFINZI® (Durvalumab), and BAVENCIO® (Avelumab); or
(iii) a CTLA-4 inhibitor comprising YERVOY (ipilimumab).

23. A method of identifying and/or isolating a T cell specific for one or more neoantigens, comprising:
(i) obtaining one or more neoantigen peptides of interest;
(ii) mixing said peptides with labile peptide loaded FLEX-T monomers;
(iii) degrading labile peptides with UV light thereby allowing for peptide exchange;
(iv) mixing peptide-exchanged monomers with streptavidin to produce MHC class I neoantigen-specific tetramers;
(v) contacting MHC class I neoantigen specific tetramers with T cells, wherein T cells specific for the one or more neoantigens will bind to the tetramer and can be identified by fluorescence; and
(vi) isolating tetramer-bound T cells.


SEQUENCE LISTING (33 sequences)

SEQ ID NO: 1 - protein, 15 aa, synthetic construct
CASEGDGRASNEQFF

SEQ ID NO: 2 - protein, 15 aa, synthetic construct
CASIRLRGNTDTQYF

SEQ ID NO: 3 - protein, 17 aa, synthetic construct
CASRYHSGQGLQETQYF

SEQ ID NO: 4 - protein, 15 aa, synthetic construct
CASSRPWELQETQYF

SEQ ID NO: 5 - protein, 18 aa, synthetic construct
CASSLVGSGRGGTDTQYF

SEQ ID NO: 6 - protein, 13 aa, synthetic construct
CASAPGGNSPLHF

SEQ ID NO: 7 - protein, 13 aa, synthetic construct
CASSPGPNSPLHF

SEQ ID NO: 8 - protein, 15 aa, synthetic construct
CAREGDGGASNEQFF

SEQ ID NO: 9 - protein, 15 aa, synthetic construct
CARGGDGRASNEQFF

SEQ ID NO: 10 - protein, 13 aa, synthetic construct
CAGSGAGSYQLTF

SEQ ID NO: 11 - protein, 12 aa, synthetic construct
CAEGSSASKIIF

SEQ ID NO: 12 - protein, 16 aa, synthetic construct
CAMREGKGGGYNKLIF

SEQ ID NO: 13 - protein, 14 aa, synthetic construct
CASSVSGVTDTQYF

SEQ ID NO: 14 - protein, 15 aa, synthetic construct
CASSLVGGYQETQYF

SEQ ID NO: 15 - protein, 18 aa, synthetic construct
CASSFRTGDFSGANVLTF

SEQ ID NO: 16 - protein, 14 aa, synthetic construct
CASSSPGISYEQYF

SEQ ID NO: 17 - protein, 13 aa, synthetic construct
CASSFGMNTEAFF

SEQ ID NO: 18 - protein, 13 aa, synthetic construct
CAISPGTYYGYTF

SEQ ID NO: 19 - protein, 14 aa, synthetic construct
CASSPRDSNYGYTF

SEQ ID NO: 20 - protein, 15 aa, synthetic construct
CASSVSVGVTDTQYF

SEQ ID NO: 21 - protein, 13 aa, synthetic construct
CASSLTAGRALHF

SEQ ID NO: 22 - protein, 13 aa, synthetic construct
CASSTGGNSPLHF

SEQ ID NO: 23 - protein, 16 aa, synthetic construct
CAENDSYGGATNKLIF

SEQ ID NO: 24 - protein, 16 aa, synthetic construct
CAMSTAGGTSYGKLTF

SEQ ID NO: 25 - protein, 13 aa, synthetic construct
CAVLIQGAQKLVF

SEQ ID NO: 26 - protein, 15 aa, synthetic construct
CAYRTLHSGNTPLVF

SEQ ID NO: 27 - protein, 13 aa, synthetic construct
CAAKMNRDDKIIF

SEQ ID NO: 28 - protein, 11 aa, synthetic construct
CALVGFNKFYF

SEQ ID NO: 29 - protein, 17 aa, synthetic construct
CAMIPPPSVGFGNVLHC

SEQ ID NO: 30 - protein, 15 aa, synthetic construct
CAVEGGGTSYGKLTF

SEQ ID NO: 31 - protein, 15 aa, synthetic construct
CAASDDSGGYQKVTF

SEQ ID NO: 32 - protein, 10 aa, synthetic construct
SSCMCGMNRR

SEQ ID NO: 33 - protein, 9 aa, synthetic construct
VVGAVGVGK